(19) United States
(12) Patent Application Publication
(10) Pub. No.: US 2011/0064815 A1
(43) Pub. Date: Mar. 17, 2011

(54) SILICIA FOR THE INHINITION OF A PROTEASE

(76) Inventors: Alexis John Toft, Cheshire (GB); Peter William Dettmar, East Yorkshire (GB); Johnathan Craig Richardson, Nottinghamshire (GB); Vicki Strugala, East Yorkshire (GB)

(21) Appl. No.: 12/989,116

(22) PCT Filed: Apr. 23, 2009
(86) PCT No.: PCT/GB2009/001055

§ 371 Date: Dec. 1, 2010

(30) Foreign Application Priority Data
Apr. 25, 2008 (GB) 0807621.8

(51) Int. Cl. A61K 9/14; A61K 33/00; A61P 1/00; B82Y 5/00

(52) U.S. Cl. 424/489; 424/724; 977/773; 977/906

(57) ABSTRACT

There is provided a silica for use to inhibit a protease. In particular there is provided a silica for treatment or prevention of a disease or condition associated with adverse protease activity or adverse proteolytic degradation within the gastrointestinal tract.

### FIELD OF THE INVENTION

The present invention relates to a silica for use as an inhibitor of a protease, silica suspensions comprising said silica, pharmaceutical compositions comprising said silica and silica suspensions and uses thereof.

### BACKGROUND TO THE INVENTION

Aspartic proteases are a group of proteolytic enzymes that are active between pH 1.5-5.5. They are characterised by the presence of two aspartic acid groups in the enzyme active site which function as general acid-base catalysts and are essential for the cleavage of peptide bonds. One of the first aspartic proteases to be characterised was human gastric pepsin, of which there are several sub-types namely Pepsin 1, 3a, 3b, 3c and gastricsin.

Pepsins are synthesised in the gastric mucosa as an inactive precursor, termed a zymogen, and following stimulation of gastric chief cells are released into the gastric lumen where they are activated by hydrochloric acid in gastric juice. The primary function of pepsin is to degrade dietary proteins and peptides into amino acid fragments suitable for absorption. The proteolytic activity of each pepsin sub-type varies with respect to gastric pH, the type of protein substrate, temperature and solute and substrate concentration. Although pepsin is active across a wide pH range, optimum proteolytic activity is usually seen at approximately pH 2-3.

Pepsin does not specifically degrade dietary protein and will indiscriminately cleave any suitable protein, peptide or glycoprotein. It will therefore degrade a range of constitutive proteins, such as collagen and elastin, as well as functional proteins, such as haemoglobin and albumin that are essential for normal physiological function. Indiscriminate degradation of these proteins, sometimes called autodigestion, is the underlying pathology of a number of disease states including dyspepsia, gastritis, ulceration and gastroesophageal reflux disease. In these disease states, the mucosa of the gastrointestinal tract is damaged by the proteolytic activity of pepsin.

The mucosal surface contains a number of constitutive and functional proteins, e.g. collagen, a large molecular weight protein, that helps maintain the integrity of the extracellular matrix, the structural framework of tissue. In the stomach, the mucosa is protected from pepsin degradation by a number of defense mechanisms including the secretion of a mucus gel layer. The mucus gel layer acts as a diffusion barrier to prevent an interaction between pepsin and the underlying mucosal surface proteins.

The mucus layer can, however, be degraded by pepsin and therefore a dynamic balance exists between mucus secretion and degradation. If this balance is disturbed, and the mucus barrier compromised, pepsin can digest the underlying epithelium and collagen resulting in tissue destruction and gastric injury. Similarly, if pepsin is refluxed beyond the oesophageal sphincter into the oesophagus, extensive tissue damage can occur as the oesophageal mucosa does not possess the protective mechanisms present in the stomach.

To prevent damage to the gastrointestinal mucosa, agents that inhibit the proteolytic activity of pepsin have been proposed.

U.S. Pat. No. 3,740,319 and U.S. Pat. No. 3,840,516 describe pepstatin, a compound extracted from culture filtrates of a strain of Streptomyces. Pepstatin was shown to inhibit the proteolytic activity of pepsin and suggested to have a preventative role in the management of gastric ulceration.

WO 01/87282 describes the use of alginates, a polysaccharide extracted from algae belonging to the order Phaeophyceae, for the inhibition of pepsin proteolytic activity. It was shown that alginates with a molecular weight of less than 400 kDa inhibited the proteolytic activity of pepsin and gastric juice activity by up to 70% and 55% respectively.

U.S. Pat. No. 3,155,575 relates to a preparation for the treatment of gastrointestinal disturbances using an aqueous suspension of an acid salt of chitosan reacted with sodium aluminate. The reacted aqueous suspension was shown to inhibit the proteolytic activity of pepsin in a ‘rat model’.

GB 1217256 describes a composition for the treatment of peptic ulcers comprising the free acid and any salt of lignosulphonic acid. Lignosulphonate was shown to reduce the proteolytic activity of pepsin against a casein substrate in an in vitro test method.

In addition, a range of organic small molecules are known to inhibit pepsin as described in GB1253317, U.S. Pat. No. 3,524,859, U.S. Pat. No. 3,459,758 and U.S. Pat. No. 3,427,305.

Qian et al (Eur Polym J, (2006), 42, 1653-1661) describe methylmethacrylate copolymer nanoparticles that reduced the activity of pepsin.

Mouecoucou et al (J Dairy Sci, (2003), 86, 3857-3865) reported that a range of plant hydrocolloids reduced the ability of pepsin to degrade peptides. They showed that xylan, gum arabic and low-methoxylated pectin inhibited the breakdown of peptides ranging in molecular weight from 1-8 kDa in the presence of pepsin. Other polysaccharides that have been shown to reduce the activity of pepsin against a protein substrate include alginate (Strugala et al, Int J Pharm, (2005) 304, 40-50), agar (Gouda and Johdka, Can J Pharm Sci, (1977), 12, 4-7), sulphated polysaccharides (Levey and Sheinfeld, Gastroenterology (1954), 27, 625-628) and oxidized starch sulphates (Namekata, Chem Pharm Bull (1962) 10, 171)

Pearson and Roberts (Clin Sci, (2001), 100, 411-417) have shown that ecabet sodium inhibited pepsin activity in gastric juice. The extent of inhibition was dependent on the pepsin sub-type.

Kratzel and Bernkop-Schnurch (Peptides (2000), 21, 289-293) have synthesised a tripeptide derivative of pepstatin A and shown that it has an inhibitory action against pepsin in vitro. They suggest that the derivative may have application in protecting peptide drugs from enzymatic degradation and may therefore be used to increase oral bioavailability.

Foster et al (Clin Sci, (1994), 87, 719-726) have shown that the polyacrylate Carbopol 934P can inhibit pepsin hydrolysis and therefore has potential as a mucosal protective agent in vivo.

Beil at al (Pharmacology, (1993), 47, 141-144) have demonstrated that bismuth subcitrate inhibits in a pH dependent manner the activity of porcine pepsin in vitro. In addition Stables et al (Aliment Pharmacol Ther, (1993), 7, 237-246) found that bismuth citrate and ranitidine bismuth citrate both inhibited pepsins 1, 2, 3 and 5.

A number of investigators have identified pepsin inhibitors from natural sources including Pacific oysters (Faisal et al, Comp Biochem and Physiol B, (1998), 121, 161-168), the roots of Anchusa strigosa (Abuereish, Phytochemistry, (1998), 48, 217-221), squash phloem exudates (Christeller et al, Eur J Biochem (1998), 254, 160), soft wheat bran (Galleschi et al, Sciences de Aliments, (1997), 17, 173-182) and panax-ginseng (Sun et al, Planta Medica, (1992), 58, 432-435).

WO 00/10527, WO 00/10528, WO 00/10529, and WO 00/10530 disclose mucoadhesive compositions comprising colloidal particles selected from silica, titania, clay and mixtures thereof. The mucoadhesive compositions of these documents are resistant to peristalsis and are used to deliver active ingredients to the gastrointestinal tract. However, there is no teachings whatsoever in these documents concerning the ability of silicas to act as an active gastrointestinal substance.

Despite the diverse range of materials that have been shown to have an inhibitory effect on the proteolytic activity of pepsin, silicas have not been mentioned in the prior art as being suitable for the inhibition of proteases.

### BRIEF SUMMARY OF THE INVENTION

In a first aspect there is provided a silica for use to inhibit a protease.

In a second aspect there is provided a silica for treatment or prevention of a disease or condition associated with adverse protease activity within the gastrointestinal tract.

In a third aspect there is provided a silica for treatment or prevention of a disease or condition associated with adverse proteolytic degradation within the gastrointestinal tract.

In a fourth aspect there is provided a silica for treatment or prevention of a disease or condition selected from the group consisting of dyspepsia, gastritis, peptic ulceration, gastroesophageal reflux disease, extra-oesophageal reflux disease, irritable bowel syndrome, rectal related inflammatory disease and inflammatory bowel disease.

In a fifth aspect there is provided a silica for use to increase intra mucin interaction.

In a sixth aspect there is provided a silica for use to increase mucus viscosity.

In a seventh aspect there is provided a silica for use to improve mucus gel properties.

For ease of reference, these and further aspects of the present invention are now discussed under appropriate section headings. However, the teachings under each section are not necessarily limited to each particular section.

### ADVANTAGES

The present invention has a number of advantages.

Pepsin (and similar proteolytic enzymes, perhaps of bacterial origin in the intestine) is an important aggressor and strongly implicated in reflux disease pathology. Inhibition of the proteolytic activity of pepsin by silicas can be an effective disease therapy by reducing the damaging potential of the reflux or luminal contents. The silicas of the present invention are capable of inhibiting proteolytic activity, such as of pepsin, and are therefore effective in therapy.

Additionally the silicas of the present invention show an ability to quench free radicals which are increased in inflammation, due to the presence of white blood cells and bacteria. The ability to quench free radicals is a measure of a materials free radical scavenging ability and thus the ability to reduce the damaging capacity in inflammatory bowel disease.

Silicas of the present invention, particularly those of small particle size (10-50 nm) are also capable of protecting epithelial cells by retarding the diffusion of pepsin across the mucosal layer (which is indicative of reduction in the accessibility of pepsin to the oesophageal mucosa which in turn will impact strongly to prevent lesions and act beneficially upon the pathology of reflux disease and dyspepsia). Since the amount of damage done to the oesophagus by pepsin is dose-dependent, any reduction in the amount of aggressor reaching the oesophagus will have a marked affect on patient symptomatology and reflux disease pathology.

The silicas of the present invention are also shown to repair compromised mucus gel and improve the gel characteristics. These findings have therapeutic potential for the treatment of ulcerative colitis and peptic ulcer in which the mucus layer is compromised and is unable to protect the underlying mucosa.

The silicas of the present invention are also capable of preventing pepsin from degrading the mucus gel and affecting its gel-forming properties. Thus, the silicas of the present invention can be protective in situations where excessive aggressors (i.e. pepsin) are present.

### DETAILED DESCRIPTION

### Silicas

According to a first aspect of the present invention there is provided a silica for use to inhibit a protease.

In the context of the present invention, an inhibitor of a protease refers to a substance which is capable of preventing the action of a protease on a substrate. In this regard, it will be understood that the occupation of the protease binding site by the inhibiting substance is not required in order to display an inhibitory effect. It will also be understood that an inhibitor is not a simple barrier material which prevent contacts between a protease and its substrate. Thus the present invention provides a silica for use to inhibit action of a protease on a substrate.

In one embodiment, there is provided a silica suitable for use as an inhibitor of a protease on a substrate wherein the silica occupies the binding site of the protease.

### Silica

Silica is the common name in the art for silicon dioxide. It may be present in a number of forms such as fumed silica, precipitated silica, amorphous silica, colloidal silica, coacervated silica, amorphous silica gel, (aqua) silica sol, hydrogel silica and xerogel silica. The silica may also be present in a liquid (soluble silicate), suspension, powder, granule or tablet form.

The silica according to the present invention may be selected from the group consisting of fumed silica, precipitated silica, amorphous silica, coacervated silica and amorphous silica gel, (aqua) silica sol, powders.

In a preferred embodiment, the silica of the present invention is amorphous silica. It is known in the art that amorphous silica can be referred to as colloidal silica. Thus, references to amorphous silica are understood to include colloidal silica.

The silica of the present invention should typically be present as nanoparticles.

Thus, in one embodiment, the silica of the present invention is present as nanoparticles. In a further embodiment, the silica has an average particle size (d50) of less than 20,000 nm. In a further embodiment, the silica has an average particle size (d50) of no greater than 18,000 nm.

In a preferred embodiment, the silica has an average particle size (d50) of less than 10,000 nm. In a further preferred embodiment, the silica has an average particle size (d50) of between about 1 nm and 5,000 nm. In a further preferred embodiment, the silica has an average particle size (d50) of less than 4,300 nm. In a further preferred embodiment, the silica has an average particle size (d50) of less than 800. In a further preferred embodiment, the silica has an average particle size (d50) of less than 180. In a still further preferred embodiment, the silica has an average particle size (d50) of between 5 nm and 100 nm. In a preferred embodiment, the silica has an average particle size (d50) of from 1 to 1,800. In a preferred embodiment, the silica has an average particle size (d50) of from 10 to 80. In a further preferred embodiment, the silica has an average particle size (d50) of less than 80. In a further preferred embodiment, the silica has an average particle size (d50) of less than 20. Silica (such as silica sols) with an average particle size (d50) in the 5 nm to about 100 nm range may remain for prolonged periods of time without significantly settling or do not aggregate to a significant extent.

In a further preferred embodiment, the silica has an average particle size (d50) of less than 100 nm. In a further preferred embodiment, the silica has an average particle size (d50) of between 5 nm and 50 nm.

In a particularly preferred embodiment, the silica has an average particle size (d50) of about 20 nm.

Table 13 and preceding data demonstrates that a silica of particle size less than 4300 nm is preferred, preferably less than 800 nm more preferably less than 180 nm, even more preferred less than 80 nm most preferred less than 20 nm.

As used herein, the term ‘average particle size’ means a particle population having a d50 of the given size. The average particle size d50 of the silica sols were measured by surface area titration and confirmed by Transmission Electron Microscopy (TEM). The average particle size of the other silica types were measured by Mastersizer. We use a Malvern Mastersizer S which uses light scattering detection to size the particles. The machine has a nominal 1000 ml sample dispersion unit with optional ultrasonic capability. We use a single lens configuration giving a size range of 0.05 μm to 880 μm with a 300RF lens and 42 element solid state detector array with two backscattering detectors. We load the sample to 15-25% obscuration and the measurement parameters are 80% pump speed, 80% stirrer speed, 50% ultrasonics and 3 minute residence time.

The silicas may have a surface areas ranging from 20 to 1200 m²/g, preferably the silicas have a surface area from 20 to 750 m²/g, more preferably the silicas have a surface area from 50 to 350 m²/g.

In a particularly preferred embodiment, the silica has an average particle size (d50) of from 10 to 80 nm and a surface area of from 50 to 350 m²/g. In this aspect, the silica is preferably in the form of a sol.

### Protease

A protease is an enzyme which conducts proteolysis. Thus, it hydrolyses the peptide bonds that link amino acids together in the polypeptide chain of proteins.

Proteases may be classified into a number of groups. Typically, they are divided into the following six groups: serine proteases, threonine proteases, cysteine proteases, aspartic acid proteases, metalloproteases and glutamic acid proteases.

Aspartic proteases include human gastric pepsin. As mentioned above, there are several sub-types of human gastric pepsin, namely pepsin 1, 3a, 3b, 3c and gastricsin.

Thus, according to one embodiment, the protease of the present invention is an aspartic acid protease. In a preferred embodiment, the protease according to the present invention is pepsin.

In a preferred embodiment, the protease according to the present invention is a mammalian pepsin.

In a preferred embodiment, the protease according to the present invention is selected from the group consisting of human pepsin, porcine pepsin, equine pepsin, murine pepsin, ovine pepsin, canine pepsin, caprine pepsin and bovine pepsin.

In a preferred embodiment, the protease of the present invention is a human pepsin.

In a preferred embodiment, the protease of the present invention is human gastric pepsin. Preferably, the protease according to the present invention is a sub-type of human gastric pepsin. Even more preferably, the protease according to the present invention is selected from the group consisting of pepsin 1, 3a, 3b, 3c and gastricsin.

In an alternative embodiment, the protease is a serine protease, preferably trypsin. In an alternative embodiment, the protease is a trypsin.

In a preferred embodiment, the protease according to the present invention is selected from pepsin and trypsin.

### Substrate

The proteases mentioned above act on a substrate. Typically, a single protease can act on a number of different substrates.

The proteases inhibited by the practice of the present invention are typically those present or originating in the gastrointestinal tract. Thus, the substrates according to the present invention are substrates typically found or originating in the gastrointestinal tract. Thus, the substrates according to the present invention include proteins found in the gastrointestinal tract.

Proteins found in the gastrointestinal tract typically include constitutive proteins, glycoproteins and functional proteins.

A constitutive protein may be considered to form part of the gastrointestinal tract and thus be considered to be inherently present in the gastrointestinal tract. A functional protein may be considered to be present in the gastrointestinal tract, but not necessarily part of the gastrointestinal tract itself.

Examples of constitutive proteins according to the present invention are collagen, mucin and elastin. Collagen forms the basement membrane of the epithelial cells lining the gut. Collagen can be degraded by a broad spectrum of proteases, such as pepsin or even specific matrix metalloproteases.

Mucus is made up of mucin glycoproteins (mucin) which consist of carbohydrate side chains on a protein backbone. Breakdown of mucin by proteases can lead to a loss of gel properties and cleavage of the glycoproteins resulting in solubilisation.

In one embodiment, the substrate of the present invention is a constitutive protein. In a preferred embodiment, the constitutive protein is collagen and/or mucin.

In one embodiment, the mucus is of gastric or colonic origin.

Examples of functional proteins include proteins present in the gastrointestinal tract but not those forming part of the gastrointestinal tract. Thus, an example of a functional protein protected by the action of the present invention is albumin.

In one embodiment, the substrate is a functional protein. In one embodiment, the functional protein is albumin.

### Silica Liquid Dose Forms

In a preferred aspect the silica provided for use in the present invention is in the form of a silica liquid dose form such as a suspension or sol, more preferably as a silica sol. Thus, in one embodiment, the silica of the present invention is present as a suspension or a silica sol. The composition of the suspension or sol is not particularly limited. However, in one embodiment, the suspension or sol comprises an alkaline medium. In an alternative embodiment, the suspension or sol comprises an acid medium.

Where the suspension or sol comprises an alkaline medium, the alkaline medium preferably comprises water and ammonia and/or sodium hydroxide.

In one embodiment the silica suspension or sol of the present invention comprises silica, water and a stabilizing alkali. In a further embodiment, the stabilizing alkali is selected from ammonia and sodium hydroxide.

In one embodiment, the silica may be present in the suspension or sol of the present invention in an amount of from about 10% to about 60% based on the weight of the suspension or sol. Preferably, the silica is present in an amount of from about 15% to about 60% based on the weight of the suspension or sol. Preferably, the silica is present in an amount of from about 20% to about 50% based on the weight of the suspension or sol. In a particularly preferred embodiment, the silica is present in the suspension or sol in an amount of about 25% or less based on the weight of the suspension or sol. In a particularly preferred embodiment, the silica is present in the suspension or sol in an amount of about 20% or less based on the weight of the suspension or sol. In a further preferred embodiment, the silica is present in the suspension or sol in an amount of about from 1 to 20% based on the weight of the suspension or sol.

In a highly preferred embodiment, the silica has an average particle size (d50) of from 1 to 180 nm and is present in the suspension or sol in an amount of about from 1 to 20% based on the weight of the suspension or sol.

The silica suspension or sol of the present invention may also comprise further components such as preservatives which prevent and/or inhibit microbial growth during storage.

In a particularly preferred embodiment the silica suspension or sol comprises silica having an average particle size (d50) of about 20 nm in an amount of about 30% based on the total weight of the suspension.

It will further be appreciated that the silica for use in the present invention may be provided in the form of a pharmaceutical composition comprising a silica or a silica suspension or a silica sol as described herein, and one or more pharmaceutically acceptable carriers, excipients, adjuvants or diluents. Thus according to a further aspect of the present invention, there is provided a pharmaceutical composition for use as an inhibitor of a protease comprising a silica and one or more pharmaceutically acceptable carriers, excipients, adjuvants or diluents.

### Applications

As mentioned herein, the present invention is suitable for the treatment of conditions and diseases associated with inappropriate proteolytic degradation within the gastrointestinal tract, such as dyspepsia, gastritis, peptic ulceration, gastroesophageal reflux disease, extra-oesophageal reflux disease, irritable bowel syndrome, and inflammatory bowel disease. Thus in one aspect there is provided a silica for treatment or prevention of a disease or condition selected from the group consisting of dyspepsia, gastritis, peptic ulceration, gastroesophageal reflux disease, extra-oesophageal reflux disease, irritable bowel syndrome, and inflammatory bowel disease. In a further aspect there is provided a silica for treatment or prevention of a disease or condition selected from the group consisting of dyspepsia, gastritis, peptic ulceration, gastroesophageal reflux disease, extra-oesophageal reflux disease, and inflammatory bowel disease.

Further, the treatment of a disease or condition associated with increased levels of free radicals present in the gastrointestinal tract is also contemplated. Thus in one aspect there is provided a silica for treatment or prevention of a disease or condition associated with increased levels of free radicals present in the gastrointestinal tract.

The above may be achieved by, for example, i) protease inhibition against a range of substrates, ii) free radical scavenging, and iii) mucus regeneration and repair.

Without being bound by theory, it may be that silicas act by strengthening the interactions between mucin molecules, perhaps by facilitating cross-linking and structural organization of biomolecules such as mucopolysaccharides and collagen. An interaction between mucin and silica may therefore improve the physiochemical properties of the mucus gel. This may allow greater protection to the underlying mucosa.

With highly purified mucin glycoproteins there were interactions with silicas such that rheological properties of the mucus solutions were vastly increased. Addition of colloidal silicas of less than 100 nm, more preferably less than 20 nm in particular resulted in very large increases in storage (G′) and loss (G″) moduli. These interactions with silicas are higher than previously seen between mucus and sodium ecabet or alginate but are in the region seen with carbopols.

### Therapy

It will be appreciated that the silica is used as therapeutic agents—i.e. in therapy applications. The term “therapy” includes curative effects, alleviation effects, and prophylactic effects.

The therapy may be on humans or animals, preferably humans.

### Pharmaceutical Compositions

In one aspect, the present invention provides a pharmaceutical composition for use in the present invention, which comprises a silica and optionally a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution/suspension.

### Combination Pharmaceutical

The compound of the present invention may be used in combination with one or more other active agents, such as one or more other pharmaceutically active agents.

By way of example, the compounds of the present invention may be used in combination with other protease inhibitors. Examples of other protease inhibitors may be found in the above references.

### Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

Depending upon the need, the agent may be administered at a dose of from 0.01 to 200 mg/kg body weight, such as from 0.1 to 150 mg/kg, more preferably from 0.1 to 100 mg/kg body weight.

By way of further example, the agents of the present invention may be administered in accordance with a regimen of 1 to 4 times per day, preferably once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The term “administered” includes but is not limited to delivery by, for example, an ingestable solution.

Thus, for pharmaceutical administration, the silicas of the present invention can be formulated in any suitable manner utilising conventional pharmaceutical formulating techniques and pharmaceutical carriers, adjuvants, excipients, diluents etc. and usually for parenteral administration. Approximate effective dose rates may be in the range from 1 to 15000 mg/day, such as from 10 to 10000 mg/day or even from 100 to 5000 mg/day depending on the individual activities of the silicas in question and for a patient of average (70 Kg) bodyweight. More usual dosage rates for the preferred and more active silicas will be in the range 200 to 2000 mg/day, more preferably, 200 to 1000 mg/day, most preferably from 200 to 500 mg/day. They may be given in single dose regimes, split dose regimes and/or in multiple dose regimes lasting over several days. For oral administration they may be formulated in tablets, capsules, solution or suspension containing from 10 to 2000 mg of compound per unit dose. Such effective daily doses will, however, vary depending on inherent activity of the active ingredient and on the bodyweight of the patient, such variations being within the skill and judgement of the physician.

### EXAMPLES

The present invention will now be described in further detail by way of example only with reference to the accompanying FIGURE in which:—

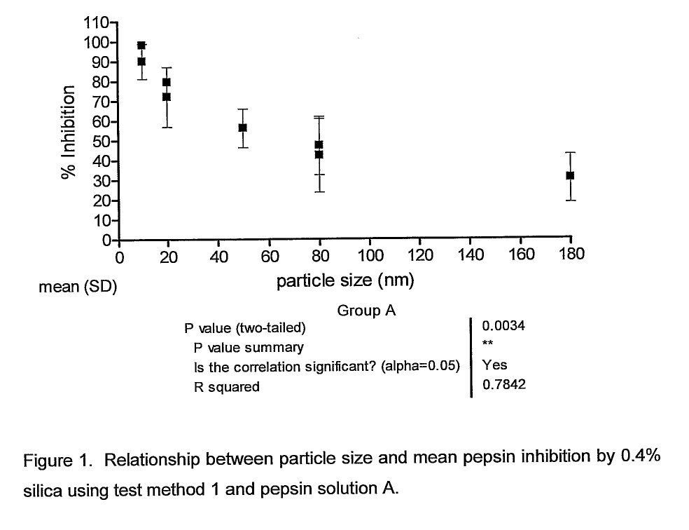

FIG. 1 shows a graph.

### MATERIALS

The silica materials used in this study were from Precision Colloids LLC, Cartersville USA and INEOS Silicas, Warrington UK. Other reagents used were obtained from standard laboratory suppliers.

The silica materials supplied in the form of a liquid or sol were diluted once in deionised water to the required concentration and shaken thoroughly. From this stock solution a volume (as specified for each test method hereafter) was taken to provide the required final concentration in the test solution (i.e. of test solution containing the pepsin and/or substrate).

The silica materials in the form of powders were dispersed in deionised water and further diluted as required and shaken thoroughly. From this stock solution a volume (as specified for each test method hereafter) was taken to provide the required final concentration in the test solution (i.e. of test solution containing the pepsin and/or substrate).

TABLE 1
Silica compounds

| | | Particle size | Initial Silica type (i.e. used | Dry solid | | |
|---|---|---|---|---|---|---|
| Example | Identity | d50 (nm) | to prepare stock solution) | content % wt | pH | Source |
| 1 | Nanosol 5080S | 80 | Sol | 50 | 8.4 | Precision Colloids |
| 2 | Nanosol 3080D | 80 | Sol | 30 | 6.0 | Precision Colloids |
| 3 | Nanosol 5050S | 50 | Sol | 50 | 8.6 | Precision Colloids |
| 4 | Nanosol 3010S | 10 | Sol | 30 | 9.2 | Precision Colloids |
| 5 | Nanosol 5050N | 50 | Sol | 50 | 6.9 | Precision Colloids |
| 6 | Nanosol 5050D | 50 | Sol | 50 | 2.1 | Precision Colloids |
| 7 | Nanosol 3020D | 20 | Sol | 30 | 4.2 | Precision Colloids |
| 8 | Nanosol 2010D | 10 | Sol | 20 | 2.5 | Precision Colloids |
| 9 | Nanosol 3080N | 80 | Sol | 30 | 7.0 | Precision Colloids |
| 10 | Nanosol 3020N | 20 | Sol | 30 | 7.6 | Precision Colloids |
| 11 | Nanosol 4020S | 20 | Sol | 40 | 8.5 | Precision Colloids |
| 12 | Lucilite | 18000 | Powder | 35 | 3.0 | INEOS Silicas (2005) |
| | (Hydrogel) | | | | | |
| 13 | Gasil 200DF | 4300 | Powder | 98 | 3.3 | INEOS Silicas (2005) |
| 14 | Gasil AB720 | 3200 | Powder | | 6.2 | INEOS Silicas (2005) |
| 15 | Gasil HP270 | 8600 | Powder | 98 | 3.0 | INEOS Silicas (2005) |
| 16 | Gasil 23D | 4600 | Powder | 97 | 6.0 | INEOS Silicas (2005) |
| 17 | Nanosol 30180D | 180 | Sol | 30 | 3.0 | Precision Colloids |
| 18 | Gasil HP270 | 800 | Suspension | 12 | | Original raw material |
| | colloidal milled | | | | | subsequently milled |
| | | | | | | to psd as indicated |
| 19 | Gasil HP270 | 1300 | Suspension | 12 | | Original raw material |
| | colloidal milled | | | | | subsequently milled |
| | | | | | | to psd as indicated |
| 20 | Kaolin | 1 | Powder | 98 | 5.0 | Sigma-USP grade |
| 21 | Aerosil | 12 | Powder | 98 | | Degussa (Evonik) |
| | (Hydrophilic) | | | | | |
| 22 | Aerosil calcined | 12 | Powder | 99.5 | | Degussa (Evonik) |
| | (Hydrophobic) | | | | | |

### pH Measurements

The pH of the silica materials supplied in sol form were measured as supplied. The pH of the silica materials supplied in powder form were determined from the 5% w/v suspension.

### Preparation of Example 18 and 19

### Colloidal Milled Silica

Equipment used for colloidal milling of Gasil HP270 to required particle size was the following:
Eiger Torrance minimill 250
182 ml Zirconium beads

The mill was assembled in accordance with the mill manufacturer's instructions using 182 ml of zirconium beads.

A Gasil HP270 slurry with a 12% w/v solid content was prepared (120 g in 100 ml demineralised water) and stirred for 10 minutes using an overhead paddle stirrer. The slurry was introduced to the mill and milled for 60 minutes at 4000 rpm. An aliquot was taken every 10 minutes for particle size distribution (PSD) analysis via Malvern Mastersizer to assess the progress of the milling.

Malvern Mastersizer method parameters were as follows:
Pump, Stirrer and ultrasonics set at 50%
2.5 minute dispersion time

### Example 22

### Calcined Aerosil

10 g Aerosil placed in a 12 cm dish and then calcined at 300° C. in a furnace for 2 hours, before being removed to a dessicator to cool.

### Pepsin Solutions

Pepsin (EC.3.4.23.1) was in the form of:

A) Porcine pepsin A (Sigma P-7012) with a specification of 2500-3500 units/mg protein. Pepsin was dissolved in 0.01M HCl (pH 2.2) to a concentration of 0-100 μg/ml.
B) Human gastric juice diluted in 0.01M HCl (to a concentration equivalent to 0-100 μg/ml of porcine pepsin).
C) Purified human pepsin 3 diluted in 0.01M HCl (to a concentration equivalent to 0-100 μg/ml of porcine pepsin).
D) Porcine pepsin A (Sigma P-7012) with a specification of 2500-3500 units/mg protein. Pepsin was dissolved in glycine/HCl buffer pH 2 to a concentration of 1 mg/ml.
E) Porcine pepsin A (Sigma P-7012) with a specification of 2500-3500 units/mg protein. Pepsin was dissolved in 0.01 M HCl to a concentration of 3 mg/ml

### Methods

Test Method 1—Pepsin Inhibition by Silica with Collagen Substrate

Pepsin activity was detected using an Azocoll assay based on the methods of Moore (1969) Anal Biochem. 32:122-127; Chavira et al. (1984) Anal Biochem. 136:446-460 and Will et al. (1984) Clin Chem. 30:707-711. This method assesses the inhibitory effect of a test substance on collagenolytic activity. The collagenolytic activity of pepsin is determined using the Azocoll digestion assay. Azocoll is a commercially available azo dye labelled collagen Type I substrate derived from bovine hide. In the presence of pepsin the red azo dye is liberated from the collagen and the resulting colour change can be measured and correlated with collagenolytic activity.

The collagen substrate was the azo-dye labelled Type I collagen, Azocoll (Calbiochem 194933) with a specification of >100 mesh. Azocoll was dissolved in pH 2.0 glycine/HCl buffer to a concentration of 0.25% and continually agitated using a magnetic follower to prevent sedimentation.

The pepsin inhibition by the silica was then measured as follows:

For each test substance (silica) three mixtures were prepared in tubes consisting each of 200 μl of the test substance (silica) mixed with 200 μl of pepsin solution of either 0, 50 or 100 μg/ml concentration (to give a final concentration of pepsin 0, 25, 50 μg/ml). Pepsin solutions A, B and C were used.

1000 μl of Azocoll solution was added to each tube and thoroughly mixed. The tubes were incubated at 37° C. for 2 hours with shaking (1200 rpm) and frequent inversion to disturb any sedimentation. Tubes were then centrifuged at 4000 rpm for 20 min. After centrifugation, 200 μl of the supernatant was transferred to a microplate and the optical density (OD) measured at 490 nm (using a microplate reader). The (OD) determined at 490 nm is a measure of type I collagen breakdown due to release of soluble azo-dye.

A 5 μg/ml solution of pepstatin A was prepared in 0.01M HCl and then diluted 1:2 with 50 μg/ml pepsin standard solution to be used as the positive control. The negative control was distilled water diluted 1:2 with 50 μg/ml pepsin standard solution.

The percentage inhibition of pepsin activity at 50 μg/ml pepsin was calculated against a calibration (cal) curve using Formula 1:

pepsin inhibition=(OD_{cal}−ODₜₑₛₜ)/(OD_{cal}×100) Formula 1

### Where:

OD_{cal}=OD value determined from the calibration curve at concentration 50 μg/ml
ODₜₑₛₜ=OD value determined from the test sample of concentration 50 μg/ml

The percentage inhibition of pepsin activity against a collagen substrate by the silica (examples 1-22) was determined and the data presented in Table 2, 3, 4 and 13.

Test Method 2—Pepsin Inhibition by Silicas with Succinyl Albumin as Protein Substrate

Pepsin activity was detected using the N-terminal assay of Hutton et al (1986) Biochem Soc Trans. 14:735-736 and detailed in Strugala et al (2005) Int J. Pharm. 304:40-50. The N-terminal assay, using pepsin as the proteolytic enzyme (relevant to dyspepsia) and succinyl albumin as the protein substrate, is a colorimetric method that detects newly formed N-terminals when a protein substrate is digested.

Protein substrate was the succinyl albumin (not commercially available), which was prepared as follows: Bovine serum albumin (fraction V) was dissolved in phosphate buffered saline pH 7.5 at a concentration of 0.2 mg/ml and constantly mixed using a magnetic stirrer. Succinic anhydride (0.014 mg/ml) was added very slowly while maintaining pH at pH 7.5 with dropwise addition of 2M NaOH. The mixture was exhaustively dialysed against deionised water and freeze dried. Succinyl albumin was then dissolved in 0.01M HCl to a concentration of 10 mg/ml and pH adjusted to pH 2.2 using dropwise addition of 1M HCl until the substrate was in solution.

For each test substance (silica) three mixtures were prepared consisting each of 10 μl of the test substance (silica) mixed with either one of 10 μl of pepsin solution of 0, 50 or 100 μg/ml concentration, to give a final concentration of pepsin 0, 25, 50 μg/ml. Pepsin solutions A, B and C were used.

A test blank was also prepared with 10 μl test substance only in which 10 μl of 100 μg/ml pepsin was to be added after addition of NaHCO₃ in order to account for the conflict interference in the assay by test substance.

50 μl of succinyl albumin solution was added and incubated at 37° C. for 30 min with shaking (600 rpm).

Pepsin activity was quenched by addition of 50 μl 4% NaHCO₃. Colour was developed by addition of 50 μl of 1% trinitrobenzene sulphonic acid with incubation at 50° C. for 10 min. The reaction was stopped by addition of 50 μl 10% sodium dodecyl sulphate and 25 μl 1M HCl.

The optical density (OD) at 405 nm was measured, the OD (405 nm) for the relevant test blank was subtracted from OD (405 nm) of the test standard curve. The OD (405 nm) of test substance with 0 μg/ml pepsin was normalised to an OD of 0.000 at 405 nm.

The percentage inhibition of pepsin activity at 50 μg/ml pepsin was calculated with Formula 1 and data presented in Table 13.

### Test Method 3—Recovery and Protection of Degraded Mucus by Silicas Measured by Rheological Parameters and Size-Exclusion Chromatography.

A series of methods exist to determine mucolytic activity of a solution. These include viscometry, rheology, gel filtration and polyacrylamide gel electrophoresis to monitor mucin turnover.

Substrate was the native mucus gel scrapped from pig stomachs (obtained from an abattoir). Mucus is made up of mucin glycoproteins (GP) which consist of carbohydrate side chains on a protein backbone. Breakdown of mucus leads to loss of gel properties and cleavage of the GP molecule resulting in its solubilisation and a decrease in molecular weight.

An in vitro model of digestion of mucus gel was set up with a tube containing approximately 1 g native pig gastric mucus mixed with 5 ml of the test solution and maintained at 37° C. From this mixture, 1 ml was sampled at each time point with fresh test solution used to replace the 1 ml sampled.

Test solutions were:

Pepsin solution D
Pepsin solution D+silica

1 ml of test solution was sampled at 0, 4, 8, and 24 hours with replacement by fresh test solution. The condition of the mucus gel was assessed visually and rheologically and the breakdown profile of the GP solubilised was determined by size-exclusion column chromatography using Sepharose CL-2B (40×1 cm column). 150 μl test sample was loaded, eluted with salt azide (0.2M NaCl/0.02% NaAzide) and 48×1 ml fractions collected. Mucus GP levels in each fraction were measured by periodic acid-Schiff's (PAS) assay as described in Mantle & Allen (1978) Biochem Soc Trans. 6:601-609. Gel properties were studied using viscosity and rheological data.

### Viscosity and Rheology Data Analysis:

Gel properties were measured using oscillatory rheology with a Bohlin CVO controlled stress rheometer using Cone and Plate geometry (CP 4°/40 mm).

An amplitude sweep was performed to find the linear viscoelastic region (LVER) of the test material and the amplitude at the midpoint was then applied for a frequency sweep. Measurements were carried out at 37° C. over a range of 0.1-100 Hz frequency of oscillation.

The parameters obtained were:

G′ (G prime)—Elastic or Storage modulus and a measure of solid-like behaviour (units=Pa);
G″ (G double prime)—Viscous or Loss modulus and a measure of liquid-like behaviour (units=Pa);
δ (delta)—Phase angle and a measure of gel strength. Tan δ=G″/G′. If δ<45° then the material is a gel (G′ dominant) and the lower the phase angle the stronger the gel.

### Test Method 4—Action of Silicas in Presence of Free Radicals

The free radical generating system was hydrogen peroxide, ascorbate, FeSO₄ and EDTA. This reaction is known as Fenton reaction, and generates the hydroxyl, superoxide and ascorbate radicals. A stock solution containing 0.5 mM ascorbate, 0.5 mM FeSO₄, 0.5 mM EDTA was prepared using phosphate buffered saline (PBS) (pH 7.4) as the diluent. Immediately prior to use, 102 μl 30% H₂O₂ (9.8M) was added to 20 ml of the stock solution to initiate free radical production (the vessel is protected from light). A standard curve was prepared containing 0, 2.5, 5, 7.5 and 10 mM H₂O₂.

100 μl of 2-deoxy-D-ribose (30.8 mM) in PBS was added to 1000 μl of free radical reaction mixture (final concentration 2.8 mM).

The positive control for this assay was 100 μM Propyl Gallate (PG). A negative control was Millipore water (or diluent of test substances).

1000 μl of standard/sample/control was added to a labelled test tube followed by addition of 100 μl of 30.8 mM deoxyribose solution mix well. After incubation at 37° C. for 1 hour in a water bath with shaking, 1000 μl 1% thiobarbituric acid solution and 1000 μl 2.8% trichloroacetic acid solution were added. Heat at 100° C. for 15 minutes in a dry block heater, then cool the tubes. 2000 μl butan-1-ol was added then centrifuged at 4000 g for 2 minutes, the organic upper layer was decanted into a disposable cuvette and OD 532 nm read using a spectrometer.

### Calculations:

% Inhibition=(OD_{cal}−ODₜₑₛₜ)/OD_{cal}×100

### Where:

OD_{cal}=free radical activity determined from calibration curve at 5 mM H₂O₂
ODₜₑₛₜ=free radical activity determined from the test sample at 5 mM H₂O₂

### Test Method 5—Barrier Properties of Silicas Against Pepsin Diffusion

The in vitro diffusion of pepsin was measured using a Franz cell model. The Franz-type diffusion cell is an established technique to evaluate diffusion and drug delivery and was developed by Dr T. Franz. The Franz cell is popular in the dermal and transdermal fields to measure diffusion of topical drugs across skin but is used for a wide range of applications including buccal and oral absorption.

The dimensions of the Franz cell used in this study were:
Donor chamber: 1.5 ml
Membrane: Millipore PTFE membrane soaked in octanol, 0.45 μm pore size
Receptor chamber: 5 ml
Aperture: 9 mm diameter
Area for diffusion: 63.6 mm²

The Franz cell was maintained at 37° C. using a thermostatically controlled heating block with built in magnetic stirrer plate.

Detection of the compound of interest in the receptor chamber was by continual closed system UV spectrometry using an HPLC pump (1 ml/min) and detector with output to a chart recorder and response measured in mm.

The receptor chamber was filled with 0.01 M HCl and the membrane clamped in place. 500 μl of pepsin solution E was applied to the donor chamber. Appearance of pepsin in the receptor chamber was detected by absorbance at a wavelength of 280 nm (A280) over 30 minutes. The influence of silica on pepsin diffusion was assessed by application of a 0.1 ml dose to the membrane prior to application of the pepsin dose.

The sections of the Franz cell relate to the following in vivo components of the gastro-oesophageal reflux model:
Donor chamber: Oesophageal lumen representing refluxate
Membrane: Oesophageal squamous cell membrane
Receptor chamber: Oesophageal cell cytoplasm

Percentage retardation of diffusion was calculated from the mean response at 30 minutes using the formula:

(response of control−response of test)/response of control×100

### Test Method 6—Trypsin Activity Assay

Trypsin activity was measured using a continuous rate spectrophotometric assay using the substrate benzoyl-L-arginine ethyl ester (BAEE) at pH 7.6. Cleavage of the arginine residue generates a new product which is detectable at 253 nm. Absorbance at 253 nm was monitored over time at 30° C. and the maximal rate of hydrolysis calculated.

Trypsin (EC 3.4.21.4) was type I bovine pancreatic trypsin (Sigma T8003). A solution of 500 U/ml trypsin diluted in 1 mM HCl was used.

Substrate was Nα-Benzoyl-L-arginine ethyl ester hydrochloride (BAEE) (Sigma B4500). A solution of 0.25 mM in 67 mM sodium phosphate buffer (pH 7.6) was prepared.

Positive control was soybean trypsin inhibitor (Sigma 93618) at 500 U/ml diluted in 67 mM sodium phosphate buffer (pH 7.6).

3000 μl of BAEE solution was pipetted into a cuvette and equilibrated to 30° C.

Absorbance at 253 nm was monitored by UV spectrometry until stable. 200 μl of test solution was added with immediate mixing by inversion. Absorbance at 253 nm was recorded for 5 minutes. Change in Absorbance at 253 nm per second (ΔA253 nm/s) was calculated.

Test conditions were 100 μl trypsin (500 U/ml)+100 μl of either:
1) 1 mM HCl (enzyme alone)
2) Soybean trypsin inhibitor (500 U/ml)
3) Silica solution

Background from the silica was assessed by using 100 μl silica+100 μl 1 mM HCl (without enzyme).

### Calculation

% trypsin inhibition=((ΔA253 nm/s trypsin−ΔA253 nm/s test)+ΔA253 nm/s trypsin)×100

### Results

TABLE 2
Porcine Pepsin inhibition by 0.4% silica (in assay
reaction mixture) with collagen substrate

| | Initial Silicas type | | Test method 1 |
|---|---|---|---|
| | (i.e. used to | Silicas particle | Mean pepsin |
| Example No | prepare stock solution) | Size d50 (nm) | inhibition (%) |
| Water | | | 0 |
| 12 | Powder | 18000 | 16 |
| 13 | Powder | 4300 | 6 |
| 19 | Suspension | 1300 | 93 |
| 18 | Suspension | 800 | 87 |
| 17 | Sol | 180 | 30 |
| 2 | Sol | 80 | 42 |
| 1 | Sol | 80 | 47 |
| 3 | Sol | 50 | 56 |
| 10 | Sol | 20 | 72 |
| 7 | Sol | 20 | 79 |
| 21 | Powder | 12 | 97 |
| 22 | Powder | 12 | 94 |
| 4 | Sol | 10 | 90 |
| 8 | Sol | 10 | 98 |
| 20 | Powder | 1 | 100 |
| Pepstatin | | | 95 |
| (1.7 μM) | | | |

Pepsin inhibition determined by test method 1 at pH 2.2 and 0.4% silica in final reaction mixture using pepsin solution A.

Data presented in Table 2 shows the effect of silica particle size on pepsin inhibition against a collagen substrate, i.e. a silica of particle size less than 4300 nm is preferred, more preferably less than 80 nm. Preferably the silica is dosed in the form of a sol. This is demonstrated graphically in FIG. 1.

TABLE 3
Porcine Pepsin inhibition as a function of % silica in assay reaction
mixture with collagen substrate using pepsin solution A

| Example | Silicas PSD | Silicas Concentration | Test Method 1 Mean |
|---|---|---|---|
| No | d50 (nm) | % Silica by weight | Pepsin inhibition (%) |
| 4 | 10 | 0.1 | 32 |
| 4 | 10 | 0.3 | 60 |
| 4 | 10 | 0.4 | 90 |
| 4 | 10 | 1 | 98 |
| 10 | 20 | 0.1 | 36 |
| 10 | 20 | 0.3 | 65 |
| 10 | 20 | 0.4 | 72 |
| 10 | 20 | 1 | 102 |
| 2 | 80 | 0.1 | 0 |
| 2 | 80 | 0.4 | 42 |
| 2 | 80 | 1.0 | 84 |
| 8 | 10 | 0.05 | 22 |
| 8 | 10 | 0.1 | 70 |
| 8 | 10 | 0.4 | 98 |
| 18 | 800 | 0.1 | 30 |
| 18 | 800 | 0.4 | 87 |
| 18 | 800 | 1.0 | 99 |
| 19 | 1300 | 0.1 | 37 |
| 19 | 1300 | 0.4 | 93 |
| 19 | 1300 | 1.0 | 100 |
| 20 | 1 | 0.05 | 26 |
| 20 | 1 | 0.1 | 70 |
| 20 | 1 | 0.4 | 100 |
| 21 | 12 | 0.05 | 14 |
| 21 | 12 | 0.1 | 42 |
| 21 | 12 | 0.4 | 97 |

Table 3 shows that at high concentrations of silica, complete inhibition of pepsin activity with a collagen substrate can be achieved. Preferably, the % silica concentration is above 0.1%, more preferably above 0.4% but preferably less than 2%. These values are final concentration of silica in the assay reaction mixture, and not what is added and thus may not be relevant as a therapeutic dose which may be higher.

TABLE 4
Human Pepsin inhibition by 0.4% silica with collagen
substrate (Test Method 1, pepsin solution A, B and C)

| Example | Silicas particle | (A) Porcine | (B) Human | (C) Human |
|---|---|---|---|---|
| No | Size (nm) | Pepsin | Gastric Juice | Pepsin 3 |
| Water | — | | 0 | 0 |
| 19 | 1300 | 93 | 94 | 92 |
| 2 | 80 | 42 | 48 | 75 |
| 8 | 10 | 98 | 98 | 98 |
| Pepstatin | — | 95 | 93 | 93 |
| (1.7 μM) | | | | |

Table 4 shows that silicas are able to inhibit pepsins of human origin (human gastric juice and isolated human pepsin 3). The extent of inhibition is similar as that achieved against porcine pepsin (93%, 42% and 98% respectively).

TABLE 5
Rheological parameters of native pig gastric
mucus in presence and absence of silicas

| Sample | G′ (Pa) | G″ (Pa) | δ (°) |
|---|---|---|---|
| Mucus | 62.59 | 7.84 | 7.1 |
| Mucus + phosphate buffered saline | 29.78 | 3.38 | 6.5 |
| Mucus + 15% Example 7 | 2088.5 | 664.23 | 17.6 |
| Mucus + 15% Example 10 | 765.50 | 232.20 | 16.9 |

Table 5 demonstrates that a mixture of silica and native mucus gives a pronounced increase in the rheological properties of native gastric pig mucus (increases in G′ and G″) indicating that the gel of the native mucus changes upon addition of silicas as a synergistic interaction. The mixtures remained G′ dominant and thus true gels. The phase angle (δ) increased slightly from 7° to approximately 17° indicating that the gel was not as strong as the native mucus-mucus interactions but still in the range expected for a good mucus gel.

Typically, healthy gastric mucus has a phase angle (δ) of 7-10° whereas healthy colonic mucus is within the range of 10-15°. If the phase angle (δ) is above 20° then this would indicate a mucus layer which is too liquid-like whereas mucus of less than 7° would be considered to have too much elastic or solid-like behaviour and thus lack flow capabilities. Degraded mucus obtained by either storing for 4 days at 37° C. (Table 6) or degradation by pepsin (Table 7) of a too high phase angle (δ) of respectively 29.93 and 55.7 were shown to be lowered in the presence of silicas.

TABLE 6
Rheological parameters of degraded native pig gastric
mucus in presence and absence of silicas

| Sample | G′ (Pa) | G″ (Pa) | δ (°) |
|---|---|---|---|
| Degraded Mucus | 0.53 | 0.31 | 29.93 |
| Degraded Mucus + 1% Example 10 | 2.29 | 0.63 | 15.43 |
| Degraded Mucus + 5% Example 10 | 31.01 | 30.04 | 14.35 |
| Degraded Mucus + 10% Example 10 | 97.52 | 23.05 | 13.67 |
| Degraded Mucus + 15% Example 10 | 127.91 | 30.41 | 13.37 |

Degraded Mucus described in Table 6 was degraded first by storing it for 4 days at 37° C. To the degraded mucus, silica was then added at different dose-levels as indicated by Table 6. Weakened mucus gel models the ulcerative colitis and gastric ulcer disease states where the gel-forming capability is reduced and unable to afford protection to the underlying mucosa. Table 6 shows that addition of silica dose lead to recovery of the mucus gel and brings it within range of gel strength required for healthy mucus as indicated by a phase angle of approximately 15° and changes in the other two rheological parameters assessed (G′ and G″). The (G′) is increased dose dependently whereas the liquid-like properties (G″), as a measure of flow remain relatively constant at higher dosage of silica. The phase angle (δ) as a measure of gel strength is dose-dependently decreased approaching that of the native mucus gel. The therapeutic advantage of this is for the treatment of ulcerative colitis and peptic ulcer where the mucus layer is compromised.

TABLE 7
Rheological parameters of native pig gastric mucus after degradation by co-incubation
with pepsin (pepsin solution D) for 24 hours in presence and absence of silicas

| | Silicas | | | |
|---|---|---|---|---|
| Sample | PSD d50 (nm) | G′ (Pa) | G″ (Pa) | δ (°) |
| Mucus + Pepsin pH 2 | | 0.245 | 0.36 | 55.7 |
| Mucus + pepsin pH 2 + 1.4% Example 8 | 10 | 5.44 | 6.00 | 39.5 |
| Mucus + pepsin pH 2 + 1.4% Example 21 | 12 | 17.47 | 6.44 | 20.3 |
| Mucus + pepsin pH 2 + 1.4% Example 10 | 20 | 2.12 | 1.69 | 39.0 |
| Mucus + Pepsin pH 2 + 7.5% Example 10 | 20 | 10.50 | 6.97 | 33.6 |
| Mucus + pepsin pH 2 + 10% Example 10 | 20 | 21.21 | 9.06 | 16.7 |
| Mucus + Pepsin pH 2 + 15% Example 10 | 20 | 44.30 | 22.52 | 27.0 |
| Mucus + pepsin pH 2 + 10% Example 2 | 80 | 9.67 | 4.74 | 26.1 |
| Mucus + pepsin pH 2 + 10% Example 17 | 180 | 0.76 | 1.02 | 53.2 |
| Mucus + pepsin pH 2 + 1.4% Example 19 | 1300 | 0.41 | 0.69 | 58.9 |

Silica (co-incubated with the pepsin and mucus) was able to dose dependently protect mucus from degradation by pepsin (Example 10 in Table 7). In the absence of silica, the mucus was completely degraded by pepsin and was no longer a gel (δ>45). Incubation of mucus with silica of preferred particle size between 1-180 nm when dosed at between 1-20% was able prevent this loss of gel properties by pepsin (Table 7). This was iterated by a reduction in solubilisation of mucin glycoprotein measured in the incubation solution (Table 8). In particular, there was substantial prevention of the appearance of large molecular weight glycoprotein molecules indicating that polymeric structure and hence gel properties of the mucus gel were maintained in the presence of silica. However the ability to protect mucus from degradation by pepsin varied depending on the silica properties. Preferably silicas should be between particle size 10-180 nm and preferably dosed in the form of a silica sol and dosed at between 1-20%. Example 21 demonstrated significant inhibition of mucus degradation in the presence of pepsin (Table 7) but did not demonstrate a reduction on the appearance of large molecular weight (Table 8) contrary to the behaviour of the other silicas with small psd (<180 nm); thereby indicating a difference in mode of action between silica sols and silica in the form of Aerosil.

TABLE 8
Release of large and small molecular weight and total glycoprotein
from native mucus gel after digestion by pepsin (pepsin solution
D) in the presence of silica after 24 hours

| | Large | Small | Total |
|---|---|---|---|
| | Mwt | MWt | GP |
| Sample | GP (μg) | GP (μg) | (μg) |
| Mucus + Pepsin pH 2 | 129 | 94 | 223 |
| Mucus + pepsin pH 2 + 1.4% Example 10 | 111 | 78 | 189 |
| Mucus + pepsin pH 2 + 10% Example 10 | 0 | 13 | 13 |
| Mucus + Pepsin pH 2 + 15% Example 10 | 2 | 104 | 106 |
| Mucus + pepsin pH 2 + 10% Example 2 | 0 | 12 | 12 |
| Mucus + pepsin pH 2 + 1.4% Example 8 | 4 | 69 | 73 |
| Mucus + pepsin pH 2 + 10% Example 17 | 36 | 20 | 56 |
| Mucus + pepsin pH 2 + 1.4% Example 19 | 79 | 79 | 158 |
| Mucus + pepsin pH 2 + 1.4% Example 21 | 268 | 133 | 402 |

TABLE 9
Effect of silicas on diffusion of pepsin across a
membrane using Test Method 5 and pepsin solution E

| | Silicas Particle Size d50 | Inhibition of Pepsin |
|---|---|---|
| Sample | (nm) | diffusion (%) |
| Control | | 0 |
| Example 4 (15%) | 10 | 68 |
| Example 7 (15%) | 20 | 70 |
| Example 10 (15%) | 20 | 69 |
| Example 11 (15%) | 20 | 63 |
| Example 3 (15%) | 50 | 74 |

The silicas were all able to inhibit the diffusion of pepsin (see Table 9). This ability to stop the pepsin reaching the lower layers would be beneficial in preventing lesion development and so would be beneficial to the pathology of reflux disease and dyspepsia.

TABLE 10
Effect of silicas on free radical scavenging using Test Method 4

| | Silicas Particle size d50 | Mean Free Radical |
|---|---|---|
| Example | (nm) | Scavenging (%) |
| 12 | 18000 | 38 |
| 13 | 4300 | 38 |
| 10 | 20 | 78 |
| 7 | 20 | 73 |
| 4 | 10 | 60 |
| 2 | 80 | 68 |

The silicas were able to scavenge free radicals as shown in Table 10. This may be relevant in control of damage that may result from inflammation

TABLE 11
Trypsin inhibition by silicas at 0.09% concentration

| | Silicas particle | Mean Trypsin |
|---|---|---|
| Example No | Size (nm) | Inhibition (%) |
| Water | — | 0 |
| 19 | 1300 | 13 |
| 2 | 80 | 9 |
| 8 | 10 | 60 |
| SoyBean Trypsin | — | 93 |
| Inhibitor | | |

TABLE 12
Trypsin inhibition by Example 8 at 0.01%-0.09%

| | Mean Trypsin |
|---|---|
| Concentration | Inhibition (%) |
| 0.01% | 21 |
| 0.02% | 22 |
| 0.045% | 28 |
| 0.09% | 60 |

Silicas were able to inhibit the enzymatic activity of trypsin, which is a serine protease, (end concentration 250 U/ml). The activity was observed at pH 7.6 using test method 6 and displayed in Tables 11 and 12.

TABLE 13
Effect of silica on pepsin activity with different
substrates (collagen, protein and mucus)

| | Silicas | Silicas | Test | Test | Test |
|---|---|---|---|---|---|
| | Surface | Particle | Method 1 | Method 2 | Method 3 |
| | Area | size | Collagen | Protein | Mucus |
| Example | (m²/g) | (nm) | % Inhibition | % Inhibition | Protection |
| 12 | 800 | 18000 | 16 | 18 | ND |
| 13 | 750 | 4300 | 6 | 25 | ND |
| 10 | 150 | 20 | 72 | 27 | Y |
| 7 | 150 | 20 | 79 | 29 | ND |
| 4 | 340 | 10 | 90 | 41 | ND |
| 2 | 50 | 80 | 42 | 22 | Y |
| 1 | 50 | 80 | 47 | ND | ND |
| 3 | 70 | 50 | 56 | ND | ND |
| 8 | 340 | 10 | 98 | 12 | Y |
| 17 | | 180 | 30 | 11 | N |
| 18 | | 800 | 87 | 28 | ND |
| 19 | | 1300 | 93 | 14 | N |
| 20 | 20 | 1 | 100 | 30 | ND |
| 21 | 200 | 12 | 97 | 17 | N |
| 22 | | 12 | 94 | ND | ND |

Y (protects mucus -phase angle (δ) <45 and reduction in the appearance of large molecular weight according to Table 8), N (does not protect mucus), ND (not determined)

Test method 1 & 2 uses pepsin solution A, Test method 3 uses pepsin solution D.

Data presented in Table 13 demonstrated the highest overall performance i.e. whether silica can inhibit the action of the gastric enzyme pepsin against three clinically relevant substrates: Collagen, a component of the basement membrane and skin, protein, the building blocks of cells, and mucus the protective gel that lines the gastrointestinal tract.

Table 13 demonstrates that silicas of particle size from 10 to 80 nm and/or of a surface area of from 50 to 350 m²/g and/or in the form of a sol gave the best overall results. If the surface area is too large then penetration into active site of pepsin and/or penetration into the mucus layer may be restricted if on the other hand the surface area is too small than contact surface area between the silica and the pepsin may be inadequate to enable pepsin inhibition.

Table 13 and preceding data demonstrates that a silica of particle size less than 4300 nm is preferred, preferably less than 800 nm more preferably less than 180 nm, even more preferred less than 80 nm most preferred less than 20 nm. Preferably the silica is dosed in the form of a suspension of hydrogel (i.e. Lucilite), more preferably a suspension of sponge-type silica (i.e. Gasil-type), even more preferably as a suspension (i.e. of milled Gasil), even more preferred as a suspension of colloidal silica (Kaolin, Aerosil) most preferred in the form of a sol (i.e. Nanosol). The above silica-types may also be dosed in a powder form.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

1. A method of inhibiting a protease comprising the administration of a pharmaceutically acceptable level of silica.

2. A method for treatment or prevention of a disease or condition associated with adverse protease activity within the gastrointestinal tract comprising the administration of a pharmaceutically acceptable level of silica.

3. A method for treatment or prevention of a disease or condition associated with adverse proteolytic degradation within the gastrointestinal tract comprising the administration of a pharmaceutically acceptable level of silica.

4. A method for treatment or prevention of a disease or condition selected from the group consisting of dyspepsia, gastritis, peptic ulceration, gastroesophageal reflux disease, extra-oesophageal reflux disease, irritable bowel syndrome, rectal related inflammatory disease and inflammatory bowel disease comprising the administration of a pharmaceutically acceptable level of silica.

5. A method according to claim 1 wherein the protease is selected from the group consisting of a serine protease, a threonine protease, a cysteine protease, an aspartic acid protease, a metalloprotease and a glutamic acid protease.

6. A method according to claim 1 wherein the protease is selected from the group consisting of a serine protease and an aspartic acid protease.

7. A method according to claim 5 wherein the protease is an aspartic acid protease.

8. A method according to claim 7 wherein the aspartic acid protease is pepsin.

9. A method according to claim 8 wherein the pepsin is selected from the group consisting of human pepsin, porcine pepsin, equine pepsin, murine pepsin, ovine pepsin, and bovine pepsin.

10. A method according to claim 9 wherein the pepsin is human pepsin.

11. A method according to claim 10 wherein the pepsin is human gastric pepsin.

12. A method according to claim 11 wherein the human gastric pepsin selected from any one of pepsin 1, pepsin 3a, pepsin 3b, pepsin 3c and gastricsin.

13. A method according to claim 5 wherein the protease is a serine protease.

14. A method according to claim 13 wherein the serine protease is trypsin.

15. A method according to claim 1 wherein the silica is selected from the group consisting of fumed silica, precipitated silica, amorphous silica, coacervated silica, amorphous silica gel, (aqua) silica sol, hydrogel silica and xerogel silica.

16. A method according to claim 15 wherein the silica is amorphous silica.

17. A method according to claim 1 wherein the silica is present as nanoparticles.

18. A method according to claim 17 wherein the silica has an average particle size (d50) of less than 20,000 nm.

19. A method according to claim 18 wherein the silica has an average particle size (d50) of less than 10,000 nm.

20. A method according to claim 19 wherein the silica has an average particle size (d50) of between about 1 nm and 5,000 nm.

21. A method according to claim 20 wherein the silica has an average particle size (d50) of between 5 nm and 100 nm.

22. A method according to claim 21 wherein the silica has an average particle size (d50) of between 5 nm and 50 nm.

23. A method according to claim 1 wherein the silica has an average particle size (d50) of from 10 to 80 nm and a surface area of from 50 to 350 m²/g.

24. A method according to claim 1 wherein the protease is inhibited in respect of activity against a substrate selected from constitutive proteins found in the gastrointestinal tract, glycoproteins found in the gastrointestinal tract, functional proteins found in the gastrointestinal tract and combinations thereof.

25. A method according to claim 24 wherein the substrate is a glycoprotein found in the gastrointestinal tract or a constitutive protein found in the gastrointestinal tract.

26. A method according to claim 24 wherein the substrate is a constitutive protein found in the gastrointestinal tract.

27. A method according to claim 26 wherein the substrate is selected from collagen and mucins.

28. A method according to claim 24 wherein the substrate is a functional protein found in the gastrointestinal tract.

29. A method according to claim 28 wherein the functional protein is albumin.

30. A method according to claim 1 wherein the silica is in the form of a silica suspension.

31. A method according to claim 30 wherein the suspension is an alkaline suspension.

32. A method according to claim 31 wherein the suspension comprises water and an alkali medium selected from ammonia or sodium hydroxide.

33. A method according to claim 30 wherein the silica is present in the suspension in an amount of from about 10% to about 50% by weight of the suspension.

34. A method according to claim 30 wherein the silica is present in the suspension in an amount of from about 15% to about 45% by weight of the suspension.

35. A method according to claim 34 wherein the silica is present in the suspension in an amount of less than about 25% by weight of the suspension.

36. A method according to claim 30 further comprising a preservative.

37. A method according to claim 1 for use to increase intra mucin interaction.

38. A method according to claim 1 for use to increase mucus viscosity.

39. A method according to claim 1 for use to improve mucus gel properties.

40. A method according to claim 37 wherein the mucin is colonic mucin or gastric mucin, or the mucus is colonic mucus or gastric mucus.

41. (canceled)

42. (canceled)

43. (canceled)

44. (canceled)
